(19) United States
(12) Patent Application Publication
(10) Pub. No.: US 2022/0401261 A1
(43) Pub. Date: Dec. 22, 2022

(54) OPHTHALMOLOGICAL SURGERY MICROSURGERY INSTRUMENTS AND METHODS OF USE IN PARS PLANA VITRECTOMY FOR INCREASING SURGICAL PRECISION AND REDUCING VITREORETINAL INSTRUMENT INSERTIONS AND REMOVALS AND RELATED TRAUMA

(71) Applicant: Harry Michael Lambert, Austin, TX (US)

(72) Inventor: Harry Michael Lambert, Austin, TX (US)

(21) Appl. No.: 17/893,577

(22) Filed: Aug. 23, 2022

Related U.S. Application Data
(63) Continuation-in-part of application No. 17/697,730, filed on Mar. 17, 2022.
Application No. 17/697,730 is a continuation-in-part of application No. 15/834,379, filed on Dec. 7, 2017, now abandoned.

(51) Int. Cl. A61F 9/007

(52) U.S. Cl. CPC A61F 9/00736; A61F 2009/00874

(57) ABSTRACT

Surgical apparatus for performing in pars plana vitrectomy microsurgery including a cannula having an intraocular portion that connects to an infusion tube. The intraocular portion includes fenestrations at its distal end. The intraocular portion receives fluid through the infusion tube and dispenses the fluid through the fenestrations lessening the flow at an infusion site in an eye. The surgical apparatus includes a vitreous cutter having a suction tube at one end and a shaft at another end. The cutting port cuts vitreous into smaller pieces or a laser that liquefies the vitreous. The shaft receives the cut vitreous pieces and the suction tube draws out the cut vitreous pieces from the eye. The surgical apparatus includes a vitreoretinal surgical tool having a vitreoretinal cutter having a scissor-like or forceps-like mechanism. The vitreoretinal cutter holds and/or cuts a membrane in the eye during the microsurgery.

### RELATED APPLICATIONS AND CLAIMS FOR PRIORITY

The present application claims priority from the following:

U.S. patent application Ser. No. 17/697,730 titled “OPHTHALMOLOGICAL SURGERY MICROSURGERY INSTRUMENTS AND METHODS OF USE FOR INCREASING SURGICAL PRECISION AND REDUCING VITREORETINAL INSTRUMENT INSERTIONS AND REMOVALS AND RELATED TRAUMA,” with Attorney Docket No. LAMB007US0 and filed Mar. 17, 2022 and

U.S. patent application Ser. No. 15/834,379, titled “SIDE FENESTRATED PARS PLANA INFUSION CANNULA,” with Attorney Docket No. LAMB001US0 and filed Dec. 7, 2017, which is incorporated herein by its entirety and referenced thereto.

### FIELD OF INVENTION

The present subject matter generally relates to an apparatus for performing microsurgeries. More specifically, the present subject matter relates to a surgical apparatus for performing a microsurgery such as ophthalmological surgical procedure and the like. Even more particularly, the present subject discloses and claims ophthalmological surgery microsurgery instruments and methods of use in pars plana vitrectomy for increasing surgical precision and reducing vitreoretinal instrument insertions and removals and related trauma.

### BACKGROUND OF INVENTION

Microsurgeries such as vitreoretinal surgery began in the early 1970's. The first device invented to perform vitreoretinal surgery (vitrectomy) was called a Vitreous Infusion Suction Cutter (VISC).

Dr. Robert Machemer was the inventor of the VISC and is widely known for his development of pars plana vitrectomy, a surgical procedure which has revolutionized the treatment of posterior segment eye diseases. During 1970's a single instrument that provides a sizable scleral incision such as 2.5 millimeter was used. Upon removal of that instrument, the eye would immediately collapse. In order to overcome the above problem, Dr. Conor O'Malley of Australia, invented a system, which required three small incisions of 0.9 mm or about 20 gauge, one with an infusion cannula, one with a light, and the third with a vitreous cutter. In order to use any other instruments like scissors, a laser, forceps, cautery, etc., eye surgeons or ophthalmologists had to remove one of the three main devices. Still, they couldn't readily remove the infusion or the light. With the removal of any instrument, the eye would depressurize and slightly collapse, leading to bleeding if the ophthalmologist were cutting vessels or vessels were bleeding due to causes like diabetic retinopathy.

With advent in technology, several surgical apparatuses have been developed that provide three incision systems, one infusion to keep the eye formed, another instrument a light, and another, the vitreous cutter. They have become smaller and smaller, now at the 27-gauge size. These surgical apparatuses still require the ophthalmologist to remove one instrument to insert another device.

Further, the cutting devices currently used are straight, and therefore cutting around the patient's lens can cause damage to the curved lens. A curved cutter would greatly facilitate surgery around the lens. A multifunctional instrument wound further expedites safer surgery.

Therefore, there is a need in the art to provide improved apparatuses of 19 gauge (1.0 millimeter) or smaller that are multifunctional in their purpose and limit the number of times they need to be taken in and out during the microsurgery and either be straight or various curved to meet the situation in surgery.

### SUMMARY

It is an object of the present invention to provide a surgical apparatus for performing a microsurgery such as ophthalmological surgical procedure and the like and that avoids the drawback of known apparatus/instrument.

It is another object of the present invention to provide a cannula configured for use during a vitreoretinal and ocular surgery.

It is another object of the present invention to provide a vitreous cutter for use during an ocular surgery.

It is another object of the present invention to provide a multifunctional vitreoretinal surgical tool for cutting or peeling of membranes and cauterization at the same time during the vitreoretinal and ocular surgery.

It is yet another object of the present invention to provide a multifunctional intraocular surgical tool for picking and dissecting vascularized tissue, membranes or scar tissue during the vitreoretinal and ocular surgery.

To achieve one or more objects, the present invention provides a surgical apparatus for performing microsurgery. The microsurgery comprises vitrectomy (vitreoretinal and ocular surgery) such as Rhegmatogenous Retinal Detachment, Macular Holes, Epiretinal Membranes, Retinal Transplantation, Dislocated intraocular lens (IOL), Non-Clearing Vitreous Hemorrhage, Proliferative Diabetic Retinopathy, Traction Retinal Detachment, Retinopathy of Prematurity, Pediatric Rhegmatogenous Retinal Detachment, Uveitis induced Retinal Detachment, Choroidal and Retinal Biopsy, Giant Retinal Tears, Choroidal Hemorrhage, Submacular Hemorrhage, Age-Related Macular Degeneration, Uveal Effusion Syndrome, Endophthalmitis, Intraocular Foreign Body, Open Globe rupture, Retinoschisis Retinal Detachment, Optic Pit Maculopathy, Retinal Detachment, and Proliferative Vitreoretinopathy.

The surgical apparatus includes a cannula having an intraocular portion. The intraocular portion includes fenestrations at one end and connects to an infusion tube at another end. The intraocular portion includes a tapered tip or curved tip upon which the fenestrations position. The intraocular portion receives fluid through the infusion tube and dispenses the fluid through the fenestrations. Fluid flows through the fenestrations and this lessens the flow to a single infusion site in an eye and limits potential retinal damage from a single injection point.

In one advantageous feature of the present invention, the fenestrations at the distal end of the intraocular portion include angled openings to distribute the flow of said fluid. This helps to avoid or reduce perpendicular injection of the fluid onto the eye and maximize posterior injection while minimizing potential posterior damage.

The surgical apparatus further includes a vitreous cutter having a handle. The vitreous cutter includes a suction tube at one end and a shaft at another end. The shaft includes a cutting or laser (liquifying of vitreous) port. Further, the shaft includes a light and/or a laser (cauterizing) and/or bipolar cautery at its distal end. The shaft comes in one of straight configuration, bent configuration and curved configuration.

In one advantageous feature of the present invention, the vitreous cutter operates at a cut rate greater than 7500 cuts per minute (cpm). The vitreous cutter operates using spring-driven mechanisms or dual pneumatic pumps or similarly productive systems that independently control the opening and closing of the cutter port or similar actuating device. The cutting port has a size of 19-gauge or smaller. The cutting port cuts vitreous into smaller pieces. The shaft receives the cut vitreous pieces and the suction tube draws out the cut vitreous pieces from the eye. In lieu of mechanical cutting, a laser that liquefies the vitreous is used. The “cutting” laser and the cauterizing laser would be separate wavelengths. The type treating/cauterizing the retina is different than the laser that would liquefy the vitreous depending on frequencies and need. The light and the laser aid in viewing the vitreous during cutting of the vitreous as well as lasering the retina, bleeders, etc. As the shaft provides light and laser at the end, it limits the number of times the vitreous cutter needs to be taken in and out during the vitreoretinal and ocular surgery. Most procedures can be completed with one entry into the eye with multifunctional instruments.

The surgical apparatus includes a vitreoretinal surgical tool having a vitreoretinal cutter or forceps or similar instrument. The vitreoretinal device comes in a scissor-like mechanism or forceps-like mechanism. The vitreoretinal cutter holds and/or cuts the membrane in the eye during the vitreoretinal and ocular surgery but provides other functions as well.

In one advantageous feature of the present invention, the vitreoretinal cutter cuts or peels of membranes and cauterizes when needed during the vitreoretinal and ocular surgery. This greatly decreases the time of surgery and likelihood of complications during and post vitreoretinal and ocular surgery since no instruments are removed.

The surgical apparatus further includes an intraocular pick and dissector for picking up the membrane or scar tissue in the eye. The intraocular pick and dissector include a shaft having a pick at its distal end. The pick extends and retracts into the shaft with the help of a button.

In one advantageous feature of the present invention, the intraocular pick and dissector allows to perform multiple tasks intraocularly in place of inserting multiple tools repeatedly into the eye during the vitreoretinal and ocular surgery.

Features and advantages of the invention hereof will become more apparent in light of the following detailed description of selected embodiments, as illustrated in the accompanying FIGUREs. As will be realised, the invention disclosed is capable of modifications in various respects, all without departing from the scope of the invention. Accordingly, the drawings and the description are to be regarded as illustrative in nature.

### BRIEF DESCRIPTION OF THE DRAWINGS

Further features and advantages of the present invention will become apparent from the following detailed description, taken in combination with the appended drawings, in which:

FIG. 1 illustrates an environment in which a surgical apparatus for performing a microsurgery implements, in accordance with one embodiment of the present invention;

FIG. 2 illustrates a cannula cutter, as in the prior art;

FIGS. 3A and 3B illustrates the surgical apparatus having a vitreous cutter, and a vitreoretinal surgical tool;

FIGS. 4A and 4B illustrates a side view and a cross-sectional view, respectively of the cannula;

FIG. 5 illustrates the feature of angled fenestration;

FIG. 6 illustrates a side view of the cannula, in accordance with another embodiment of the present invention;

FIG. 7 illustrates a side view of the cannula, in accordance with yet another embodiment of the present invention;

FIGS. 8A to 8D illustrate the vitreous cutter having a shaft in different configurations, in accordance with several embodiments of the present invention;

FIGS. 9A to 9C illustrate a front, a top and a rear side view, respectively of a shaft having a cutting port;

FIG. 10 illustrates a perspective view of the shaft having laser at its distal end;

FIG. 11 illustrates a perspective view of the laser connecting via laser wire;

FIG. 12 illustrates a cross-section of the shaft having the laser;

FIG. 13 illustrates a perspective view of the shaft having light at its distal end;

FIG. 14 illustrates a perspective view of the light connecting via light wire;

FIG. 15 illustrates a cross-section of the shaft having the light;

FIG. 16 illustrates a perspective view of shaft having bipolar cautery probe and light;

FIG. 17 illustrates the feature of laser connecting via laser wire and light connecting via light wire;

FIG. 18 illustrates a cross-section of the shaft having bipolar cautery probe connecting via wire and light connecting via light wire;

FIGS. 19 and 20 illustrate a shaft having lights, laser and bipolar cautery probes;

FIG. 21 illustrates a perspective view of the vitreoretinal surgical tool;

FIG. 22 illustrates a vitreoretinal cutter, in accordance with one embodiment of the present invention;

FIG. 23 illustrates the feature of vitreoretinal cutter placed in the eye during a microsurgery;

FIGS. 24 to 28 illustrate an intraocular portion having a vitreoretinal cutter, in accordance with various embodiments of the present invention;

FIG. 29 illustrates a perspective view of an intraocular pick and dissector, in accordance with one embodiment of the present invention;

FIGS. 30 and 31 illustrate a perspective and a top view, respectively of a pick extending from a shaft;

FIGS. 32 and 33 illustrate the feature of the pick extending and retracting into the shaft,

FIGS. 34 and 35 illustrate the feature of the shaft receiving the pick;

FIG. 36 illustrates a feature of the intraocular pick and dissector placed in the eye;

FIG. 37 illustrates a perspective view of an intraocular pick and dissector, in accordance with another embodiment of the present invention; and

FIG. 38 illustrates the feature of the shaft having light and laser at distal end.

It will be noted that throughout the appended drawings, like features are identified by like reference numerals.

### DETAILED DESCRIPTION OF THE EMBODIMENTS

Before the present features and working principle of a surgical apparatus is described, it is to be understood that this subject matter is not limited to the particular surgical apparatus as described, since it may vary within the specification indicated. Various features of a surgical apparatus might be provided by introducing variations within the components/subcomponents disclosed herein. It is also to be understood that the terminology used in the description is for the purpose of describing the particular versions or embodiments only, and is not intended to limit the scope of the present subject matter, which will be limited only by the appended claims. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

It should be understood that the present invention describes a surgical apparatus for performing a microsurgery. The surgical apparatus includes a cannula having an intraocular portion. The intraocular portion connects to an infusion tube. The intraocular portion includes fenestrations at its distal end. The intraocular portion receives fluid through the infusion tube and dispenses the fluid through the fenestrations lessening the flow at an infusion site in an eye. The surgical apparatus includes a vitreous cutter. The vitreous cutter includes a suction tube at one end and a shaft at another end. The cutting port cuts vitreous into smaller pieces. The shaft receives the cut vitreous pieces and the suction tube draws out the cut vitreous pieces from the eye. The surgical apparatus includes a vitreoretinal surgical tool having a vitreoretinal cutter. The vitreoretinal cutter has a scissor-like or forceps-like mechanism. The vitreoretinal cutter holds and/or cuts a membrane in the eye during the microsurgery.

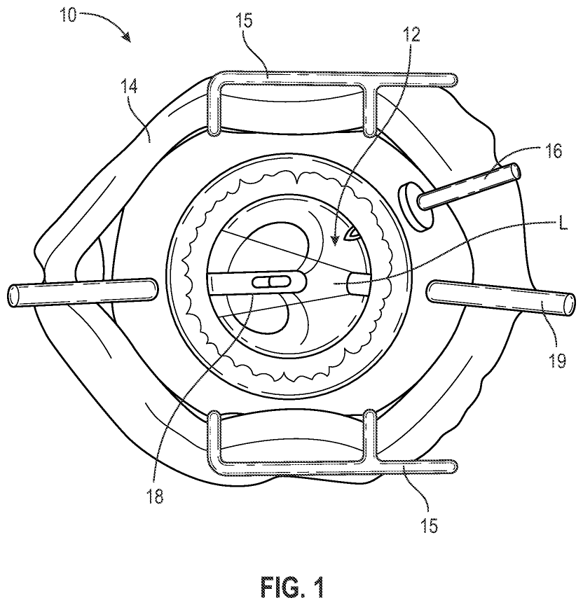

Various features and embodiments of a surgical apparatus for performing a microsurgery are explained in conjunction with the description of FIGS. 1-38.

The present invention discloses a surgical apparatus for performing a microsurgery. FIG. 1 shows an exemplary environment 10 as viewed by a surgeon using surgical apparatus 12 for performing a microsurgery on eye 14, in accordance with one embodiment of the present invention. The microsurgery includes ophthalmological surgical procedure/vitrectomy/vitreoretinal and ocular surgery such as Rhegmatogenous Retinal Detachment, Macular Holes, Epiretinal Membranes, Retinal Transplantation, Dislocated intraocular lens (IOL), Non-Clearing Vitreous Hemorrhage, Proliferative Diabetic Retinopathy, Traction Retinal Detachment, Retinopathy of Prematurity, Pediatric Rhegmatogenous Retinal Detachment, Uveitis induced Retinal Detachment, Choroidal and Retinal Biopsy, Giant Retinal Tears, Choroidal Hemorrhage, Submacular Hemorrhage, Age-Related Macular Degeneration, Uveal Effusion Syndrome, Endophthalmitis, Intraocular Foreign Body, Open Globe rupture, Retinoschisis Retinal Detachment, Optic Pit Maculopathy, Retinal Detachment, and Proliferative Vitreoretinopathy.

In order to perform the microsurgery, an eye surgeon places speculums or retractors 15 to hold eye lid 14 open, as shown in FIG. 1. The surgeon inserts cannula 16. Cannula 16 allows a fluid to go into the eye to replace vitreous. Further, the surgeon inserts vitreous cutter 18 for removing one of remove scar tissue, laser repair of retinal detachments and treatment of macular holes. The vitreous cut by the vitreous cutter 18 is removed through vitreous cutter 18. During the surgery, fiberoptic light 19 projects light L for aiding the surgeon to cut and remove the vitreous.

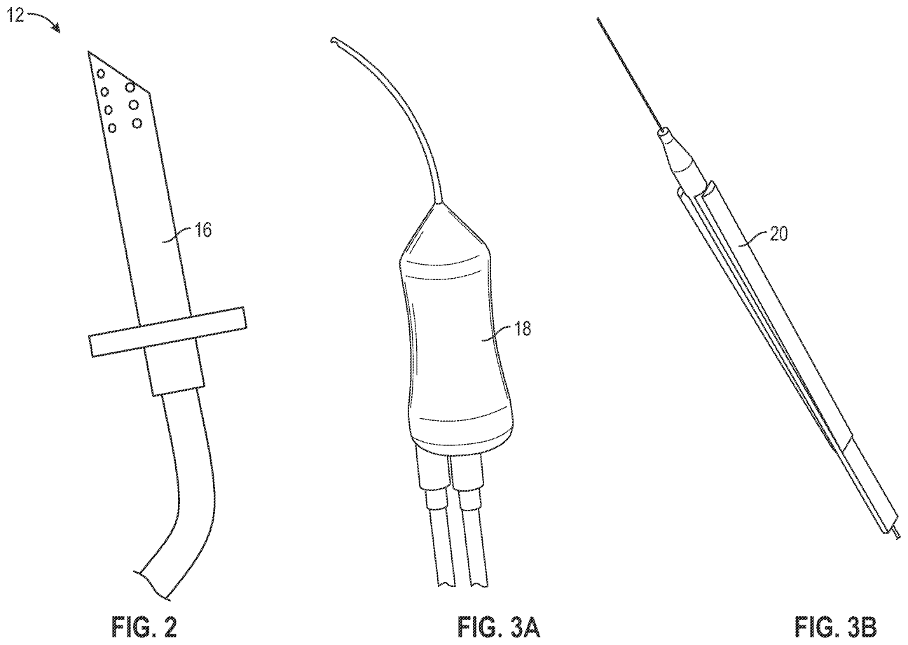

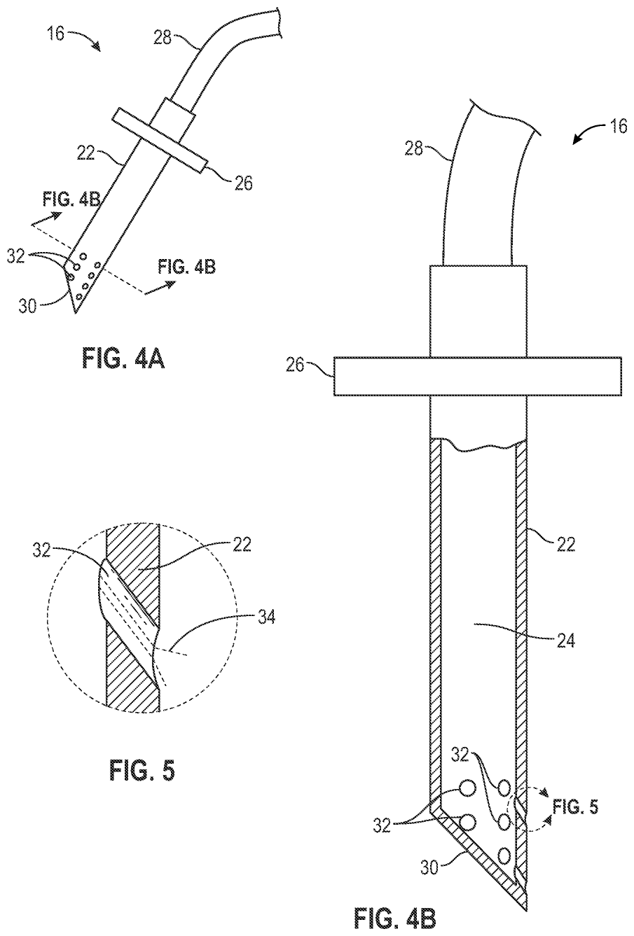

FIG. 2 shows an infusion cannula 16 and vitreous cutter 18, respectively as known in the art. FIGS. 3A and 3B show vitreous cutter 18, and vitreoretinal surgical tool or squeeze handle 20, respectively, in accordance with one embodiment of the present invention. FIG. 4A shows a side view of cannula 16 having fenestrations or openings, in accordance with one embodiment of the present invention. Cannula 16 includes intraocular portion 22. Intraocular portion 22 indicates a tube-like or syringe-like structure having interior 24 extending the entire length of intraocular portion 22. FIG. 4B shows a cross-sectional view of intraocular portion 22 having interior 24. Intraocular portion 22 encompasses guard portion 26. Guard portion 26 surrounds intraocular portion 22 and helps to operate cannula 16 by an eye surgeon or ophthalmologist. Intraocular portion 22 connects to infusion tube 28 at one end, as shown in FIGS. 4A and 4B. Infusion tube 28 receives fluid that exits through the distal end of intraocular portion 22 to remove vitreous humor or vitreous inside eye 14. In accordance with one embodiment, intraocular portion 22 presents tapered tip 30 at its distal end to prevent damage to eye 14 and safe entry to eye 14 during the procedure. FIGS. 4A and 4B show the feature of intraocular portion 22 having tapered tip 30. At the distal end, intraocular portion 22 presents plurality of fenestrations or openings 32. In one example, fenestrations 32 indicate openings or pores spread along the entire tapered tip 30. In another example, fenestrations 32 spread the entire length of intraocular portion 22 (i.e., from the distal end having tapered tip 30 to other end where infusion tube 28 connects). Here, fenestrations 32 position at equal distance or varied distance from one another. A person skilled in the art understands that fenestrations 32 can come in different shapes, sizes and numbers depending on the need without departing from the scope of the present invention. Further, each fenestration 32 includes an angled opening allowing liquid 34 to dispense or spray at a less pressure/flow. FIG. 5 shows the feature of fenestration 32 having angled opening through which liquid 34 dispenses.

In conventional cannulas, the liquid exists through the distal end of the intraocular portion having a single opening (i.e., at tapered tip 30) during the vitreoretinal and ocular surgery. This creates a “jet stream” of fluid or gas being injected directly into the eye, which can hit the opposite side of the retina and potentially damage it. In order to overcome the above problem, the presently disclosed cannula 16 presents intraocular portion 22 having fenestrations 32, through which the liquid received from infusion tube 28 is made to spread and then hit the eye. The spreading of the liquid from fenestrations 32 lessens the flow on the retina at a given infusion site and prevents the “jet stream” effect associated with the conventional cannulas. Further, angled fenestrations 32 help to avoid or reduce perpendicular injection of fluid and maximize posterior injection in side fenestrated pars plana infusion cannulas during the vitreoretinal and ocular surgery.

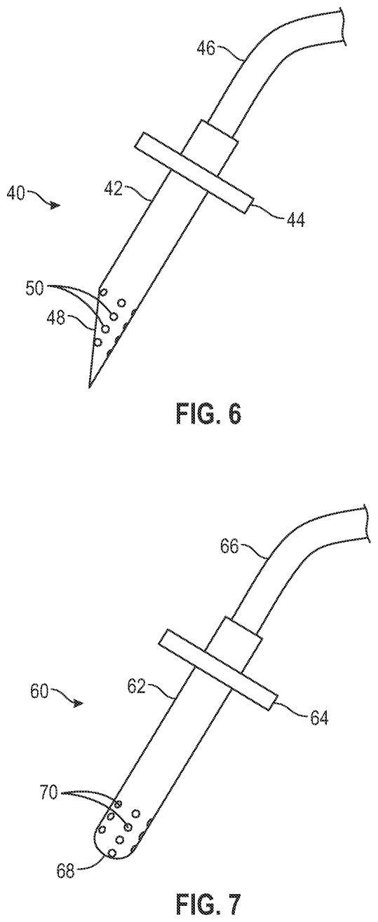

FIG. 6 shows cannula 40, in accordance with another embodiment of the present invention. Similar to cannula 16, cannula 40 includes intraocular portion 42. Intraocular portion 42 encompasses guard portion 44. Guard portion 44 surrounds intraocular portion 42 and helps to operate cannula 40 by an eye surgeon or ophthalmologist. Intraocular portion 42 connects to infusion tube 46 at one end and presents tapered tip 48 at its distal end. Here, intraocular portion 42 presents fenestrations 50 extending at tapered tip 48. The present embodiment is shown to illustrate position of fenestrations 50 only at tapered tip 48. A person skilled in the art understands that cannula 40 operates similarly to cannula 16, as explained above and prevents the “jet stream” effect associated with the conventional cannulas.

FIG. 7 shows cannula 60, in accordance with another embodiment of the present invention. Similar to cannula 16, cannula 60 includes intraocular portion 62. Intraocular portion 62 encompasses guard portion 64. Guard portion 64 surrounds intraocular portion 62 and helps to operate cannula 60 by an eye surgeon or ophthalmologist. Intraocular portion 62 connects to infusion tube 66 at one end and presents curved tip 68 at its distal end. Here, intraocular portion 62 presents fenestrations 70 at curved tip 68. A person skilled in the art understands that cannula 60 operates similarly to cannula 16, as explained above and prevents the “jet stream” effect associated with the conventional cannulas.

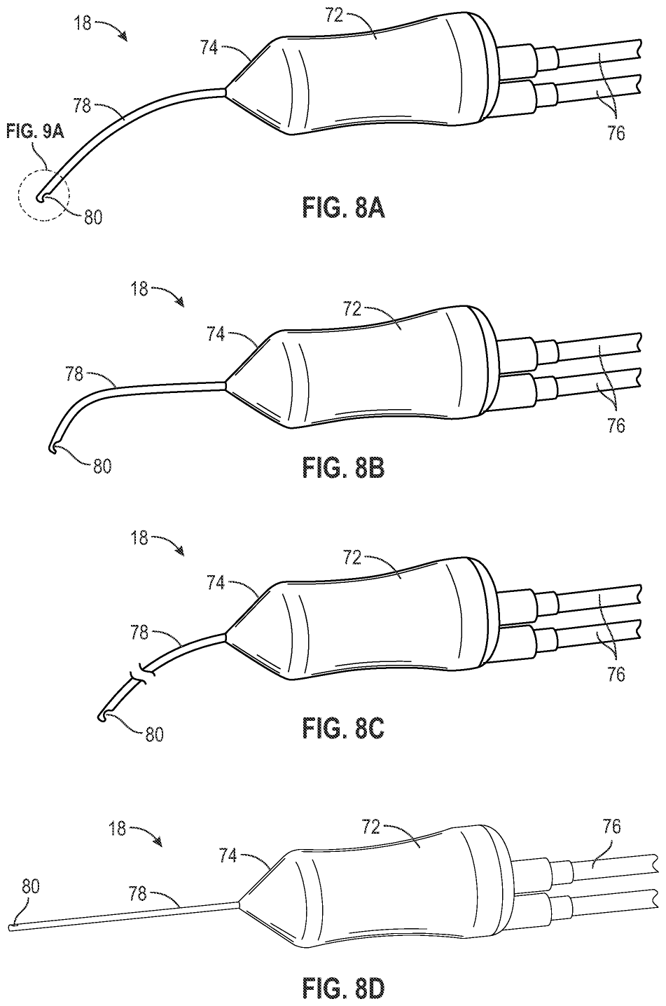

As shown in FIG. 2, surgical apparatus 12 includes vitreous cutter 18. FIG. 8A shows the feature of vitreous cutter 18, in accordance with one embodiment of the present invention. Vitreous cutter 18 presents handle 72. In one implementation, handle 72 allows the ophthalmologist to hold vitreous cutter 18 and remove scar tissue, laser repair of retinal detachments and treatment of macular holes. At one end, handle 72 includes tapered portion 74. At the other end, handle 72 connects to suction tube 76 that connects to port. Vitreous cutter 18 includes shaft or probe 78 extending from tapered portion 74 of handle 72. Shaft 78 comes in a variety of configurations such as curved, bent, elongated in straight or bent, or straight. The curved shaft 78 helps to avoid the lens and cause the cataract. FIG. 8A shows shaft 78 in a curved configuration. FIG. 8B shows shaft 78 in a bent configuration. FIG. 8C shows shaft 78 in an elongated configuration. FIG. 8D shows shaft 78 in a straight configuration. A person skilled in the art understands that shaft 78 can come in any other configuration without departing from the scope of the present invention.

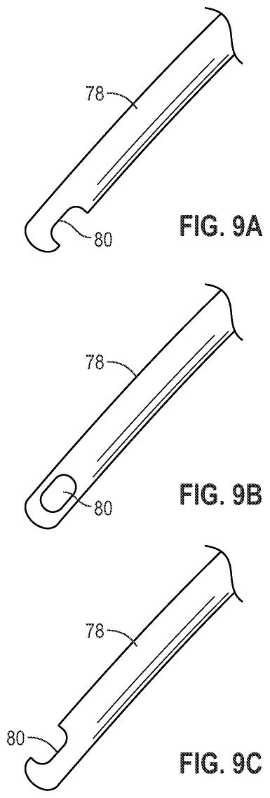

Shaft 78 encompasses a hollow structure or opening (not shown) extending the entire length of shaft 78. Shaft 78 presents cutting port 80. Cutting port 80 has a U-shaped configuration and positions at distal end 81 of shaft 78. FIGS. 9A, 9B and 9C show a front, a top and a rear side view, respectively of shaft 78 having cutting port 80, in accordance with one embodiment of the present invention. In one example, cutting port 80 has a size of 19-gauge or 1 millimeter. In another example, cutting port 80 has a size of 27-gauge. A person skilled in the art understands that cutting port 80 can come in any other shape and size without departing from the scope of the present invention.

Although FIGS. 9A, 9B and 9C show shaft 78 having a single cutting port 80, it is possible to provide more than one cutting port 80 along the length (or its distal end 81) of shaft 78 to cut the vitreous into smaller pieces and to improve the flow into cut pieces into suction tube 76. Further, a person skilled in the art understands that cutting port 80 can come any other size/dimension depending on the need. For instance, the ophthalmologist may select shaft 78 having cutting port 80 with a size of 19 gauge or smaller in a diabetic surgery, macular surgery, retinal detachment, and other procedures that require fine dissection of membranes in a traction-less environment. Here, the ophthalmologist selects shaft 78 having cutting port 80 based on a number of variables, including but not limited to, the port depth, port diameter, distance between the port and tip, external shaft diameter, and internal shaft diameter.

The presently disclosed vitreous cutter 18 is capable of operating at significantly higher cut rates of 7500 to 8000 or even more say up to 16,000 cuts per minute (cpm) using spring-driven mechanisms or dual pneumatic pumps that independently control the opening and closing of cutter port 80. Faster cutting speed helps to achieve more efficient surgery as the vitreous is being cut into smaller pieces and thus the flow is improved. Further, faster cutters result in safer vitrectomy because of the reduced traction on the retina. Furthermore, curved shaft 78 greatly alleviates damage to the eye during the pars plana vitreoretinal and ocular surgery and facilitates better removal of the vitreous and proliferative and scar tissue. A person skilled in the art understands that curved shaft 78 can be used to remove the vitreous using mechanical, ultrasound, laser or any other conventional known means of removing the vitreous.

In pars plana vitreoretinal and ocular surgery, retinal or neovascular vessels may be cut. Further, removing of the vitreous cutter after surgery from the eye instantly lowers the flow in the eye allowing ongoing bleeding or to considerably worsen, sometimes filling the eye with vision obscuring blood requiring its removal before continuing. In order to address the above problem, a conventional vitreous cutter needs to be removed and cautery has to be replaced to stop bleeding. Subsequently a laser and a light are inserted separately to treat the retina or other structure.

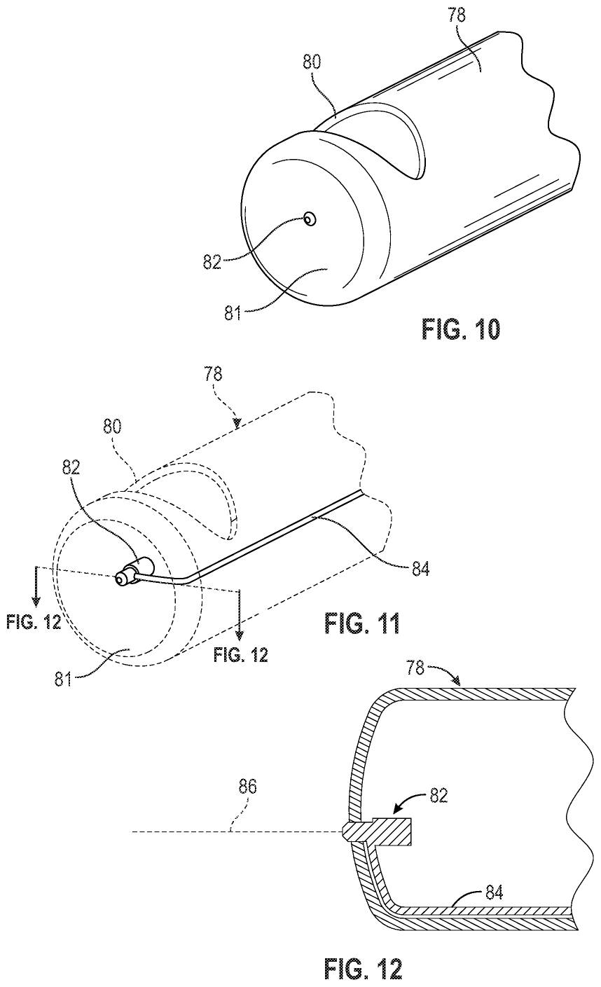

In order to overcome the above problems, the presently disclosed vitreous cutter 18 includes a laser, a light and cautery probe fitted at distal end 81 of shaft 78. This multifunctional vitreous cutter 18 with additional built-in features, such as light, laser, cautery probe and other devices allow it to be used without having to remove and insert several times during pars plana vitreoretinal and ocular surgery. FIG. 10 shows a perspective view of shaft 78 having laser 82 at its distal end 81, in accordance with one embodiment of the present invention. Laser 82 connects via laser wire 84 that draws power from a power source (not shown). FIG. 11 shows the feature of laser 82 connecting via laser wire 84. As can be seen, laser wire 84 draws and extends at the interior of shaft 78 and as such it is not visible from the outer side. This ensures laser wire 84 does not hinder operation of vitreous cutter 18 to remove scar tissue, laser repair of retinal detachments and treatment of macular holes, for example. Laser 82 draws power through laser wire 84 and projects laser 86 to treat the retina or other structure in eye 14. FIG. 12 shows a cross-section of shaft 78 having laser 82.

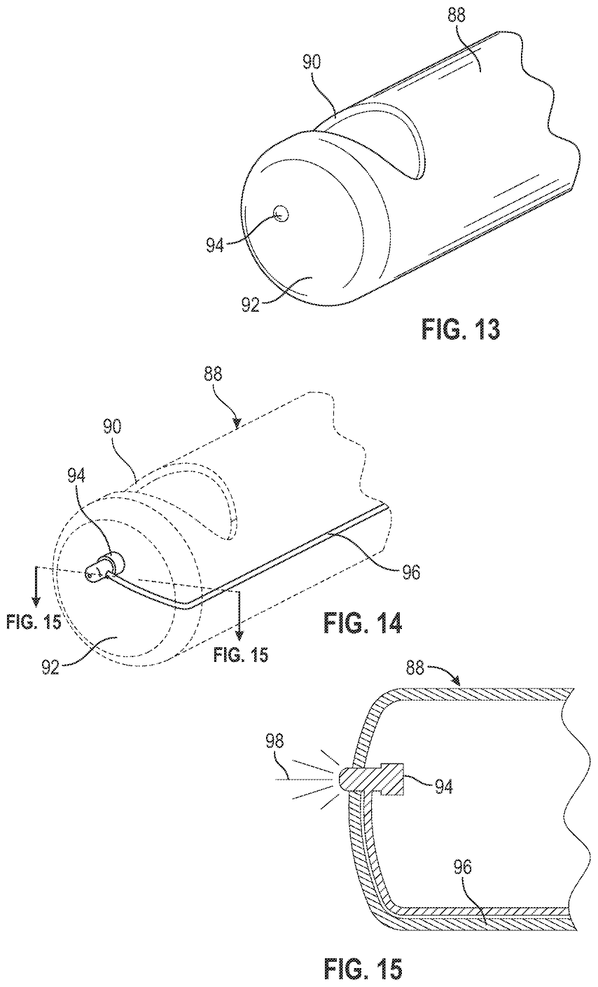

FIG. 13 shows a perspective view of shaft 88 having light 94, in accordance with another embodiment of the present invention. Here, shaft 88 encompasses cutting port 90. Shaft 88 presents light 94 at its distal end 92. Light 94 connects via light wire 96 that draws power from a power source (not shown). FIG. 14 shows the feature of light 94 connecting via light wire 96. Light 94 draws power through light wire 96 and projects light 98 to help the ophthalmologist to treat the retina or other structure in eye 14. FIG. 15 shows a cross-section of shaft 88 having light 94.

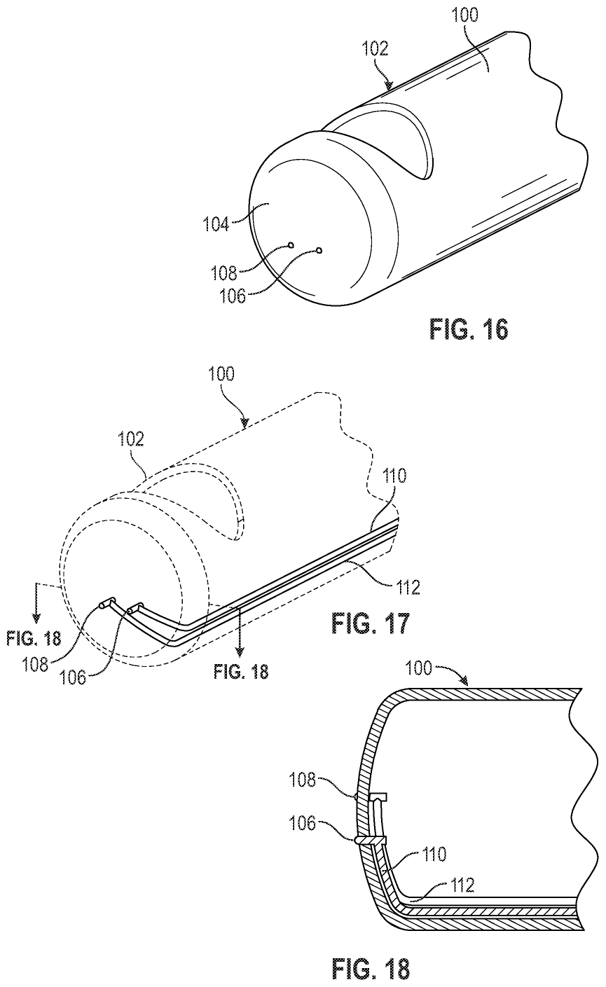

FIG. 16 shows a perspective view of shaft 100 having bipolar cautery probe 106 and light 108, in accordance with another embodiment of the present invention. Here, shaft 100 encompasses cutting port 102. Shaft 100 presents bipolar cautery probes 106 and light 108 at its distal end 104. Bipolar cautery probe 106 connects via wire 110 that draws power from a power source (not shown). Light 108 connects via light wire 112 that draws power from a power source (not shown). FIG. 17 shows the feature of laser 106 connecting via laser wire 110 and light 108 connecting via light wire 112. As can be seen, light wire 112 draws and extends at the interior of shaft 100. This ensures light wire 112 does not hinder operation of vitreous cutter 18 to remove scar tissue, laser repair of retinal detachments and treatment of macular holes. FIG. 18 shows a cross-section of shaft 100 having bipolar cautery probe 106 connecting via wire 110 and light 108 connecting via light wire 112. Here, bipolar cautery probe 106 draws power through laser wire 110 and projects laser to treat the retina or other structure in eye 14 while light 108 projects light to help the ophthalmologist to treat the retina or other structure in eye 14.

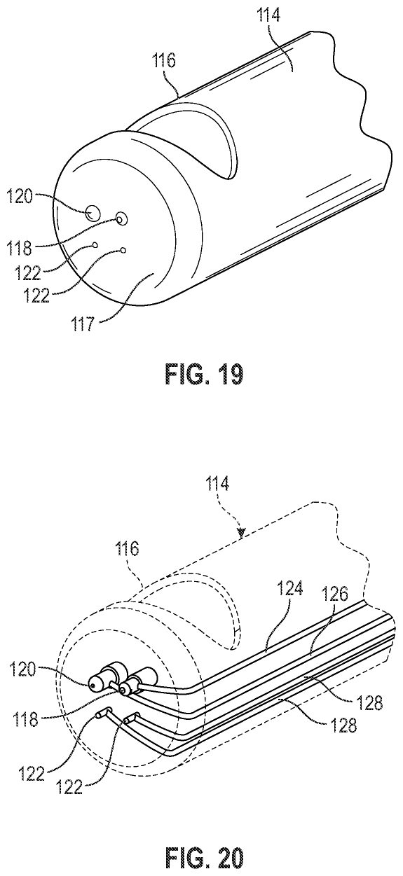

FIG. 19 shows a perspective view of shaft 114 having laser 118, light 120 and bipolar cautery probe 122, in accordance with yet another embodiment of the present invention. Here, shaft 114 encompasses cutting port 116. Shaft 114 presents laser 118, light 120 and bipolar cautery probe 122 at its distal end 117. Laser 118 connects via laser wire 124 that draws power from a power source (not shown). Light 120 connects via light wire 126 that draws power from a power source (not shown). Further, bipolar cautery probes 122 connect via probe wires 128 that draw power from a power source (not shown). FIG. 20 shows the feature of laser 118 connecting via laser wire 124, light 120 connecting via light wire 126 and bipolar cautery probe 122 connecting via probe wires 128. Here, laser 118 draws power through laser wire 124 and projects laser to treat the retina or other structure in eye 14 while light 120 projects light to help the ophthalmologist to treat the retina or other structure in eye 14. Bipolar cautery probe 122 allows for cauterization in addition to cutting during the surgery. This ensures that vitreous cutter 18 (having light, laser and cautery probe) can be used without requiring insertion of separate cautery tools. In addition, laser wire 124, light wire 126 and probe wires 128 position at the inner side of shaft 114. As such, they do not interfere with the cutting or suction operation by shaft 114 and cutting port 116.

From the above, a person skilled in the art understands that the presently disclosed vitreous cutter provides a multifunctional cutter that overcomes the need to remove vitreous cutter and insert a separate tool during the ocular surgery. This improves efficiency of the ocular surgery while potentially reducing the risk of serious damage to the eye due to removal and re-entry of additional tools during the surgery. Further, this increases safety and reduces the risks associated with certain ocular procedures during the ocular surgery.

As shown in FIG. 2, surgical apparatus 12 includes vitreoretinal surgical tool 20. Typically, vitreoretinal and ocular surgeries tend to require the use of multiple tools, which in turn requires multiple entries into the eye to change the instrument. Multiple entries of instruments into the eye risks the loss of pressure/flow and potential for haemorrhage and complications with each entry. In order to overcome the above difficulties, the presently disclosed vitreoretinal surgical tool 20 provides a single tool to cut or peel of membranes and cauterization at the same time. This limits the number of entries into the eye and greatly decreases the time of surgery and likelihood of complications during the vitreoretinal and ocular surgery.

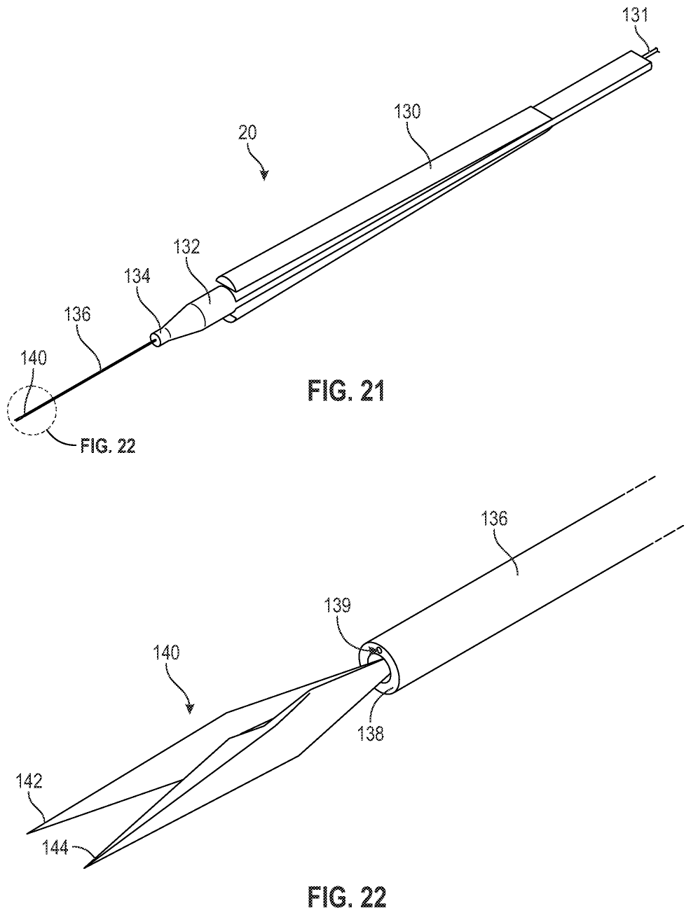

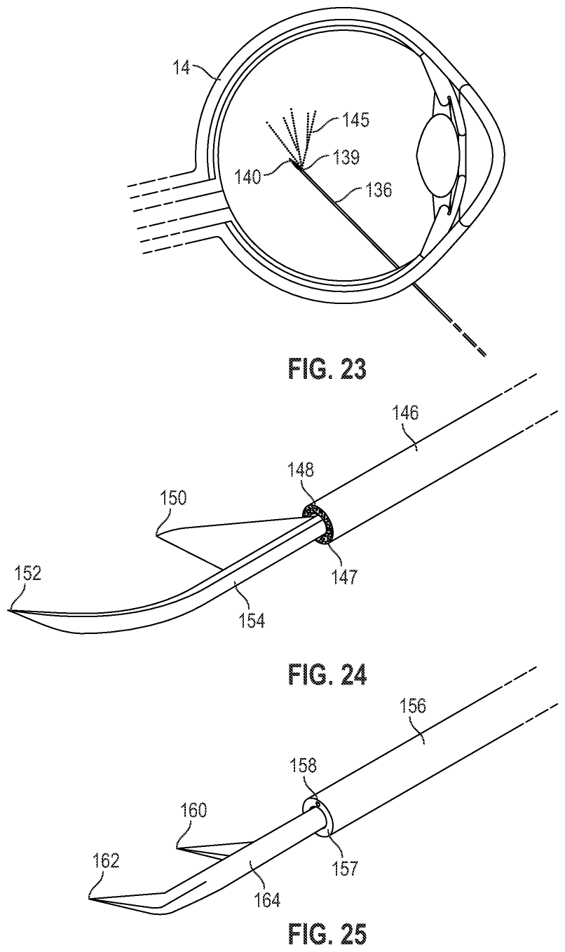

FIG. 21 shows a perspective view of vitreoretinal surgical tool 20, in accordance with one embodiment of the present invention. Vitreoretinal surgical tool 20 includes handle 130 that receives tubular section 132. Handle 130 connects to a power source and a light source 131 to provide light at the retina for operating vitreoretinal surgical tool 20. Tubular section 132 has tapered portion 134. Vitreoretinal surgical tool 20 encompasses intraocular portion 136 extending from tapered portion 134 of tubular section 132. Intraocular portion 136 presents distal end 138. At distal end 138, intraocular portion 136 encompasses light 139. Intraocular portion 136 includes vitreoretinal cutter 140. Vitreoretinal cutter 140 encompasses a scissor-like or forceps-like mechanism for holding and/or cutting of membrane in eye 14. FIG. 22 shows an exemplary vitreoretinal cutter 140, in accordance with one embodiment of the present invention. Vitreoretinal cutter 140 includes first cutter 142 and second cutter 144. Each of first cutter 142 and second cutter 144 indicates a blade for holding and/or cutting of membrane in eye 14. FIG. 23 shows a feature of vitreoretinal cutter 140 placed in eye 14. Here, vitreoretinal cutter 140 inserts in eye 14. Upon placing vitreoretinal cutter 140, light 138 emits light 145 for aiding the ophthalmologist during the vitreoretinal and ocular surgery. The ophthalmologist engages handle 130 to actuate intraocular portion 136 for operating vitreoretinal cutter 140 to cut and/or remove the membrane in eye 14.

In one implementation, first cutter 142 acts as a positive pole for cauterization and second cutter 144 acts as a negative pole for cauterization. When actuated with the help of handle 130, first cutter 142 and second cutter 144 cut the membrane and cauterize at the same time. This ensures the membrane is cut and cauterized with the same instrument without necessitating the removal and re-entry of multiple tools during the surgery. This reduces the time taken for surgery and the likelihood of complications taking in and out of multiple instruments during the surgery.

A person skilled in the art understands that vitreoretinal surgical tool 20 can also be used in proliferative vitreoretinopathy (PVR) procedures and diabetic retinopathy and other similar surgeries, such as those involving tumors.

FIG. 24 shows intraocular portion 146 having vitreoretinal cutter 150, in accordance with another embodiment of the present invention. Here, intraocular portion 146 presents distal end 147. At distal end 147, intraocular portion 146 encompasses light ring 148 having a plurality of lights. Intraocular portion 146 includes vitreoretinal cutter 150. Vitreoretinal cutter 150 encompasses a curved scissor-like mechanism for holding and/or cutting of membrane in eye 14. Vitreoretinal cutter 150 includes first cutter 152 and second cutter 154. Here, first cutter 152 indicates a curved blade having positive pole for cauterization and second cutter 154 indicates a curved blade having negative pole for cauterization. Here, vitreoretinal cutter 150 draws power from power source and a light source 131 and helps to hold and/or cut membrane in eye 14 during the vitreoretinal and ocular surgery.

FIG. 25 shows intraocular portion 156 having vitreoretinal cutter 160, in accordance with another embodiment of the present invention. Here, intraocular portion 156 presents distal end 157. At distal end 157, intraocular portion 156 encompasses laser 158 used for treating/bonding the tissue in eye 14. Intraocular portion 156 includes vitreoretinal cutter 160. Vitreoretinal cutter 160 encompasses a vertical scissor-like mechanism for holding and/or cutting of membrane in eye 14. Vitreoretinal cutter 160 includes first cutter 162 and second cutter 164. Here, first cutter 162 indicates a vertical blade having positive pole for cauterization and second cutter 164 indicates a vertical blade having negative pole for cauterization. Here, vitreoretinal cutter 160 draws power from power source and a light source 131 and helps to hold and/or cut membrane in eye 14 during the vitreoretinal and ocular surgery.

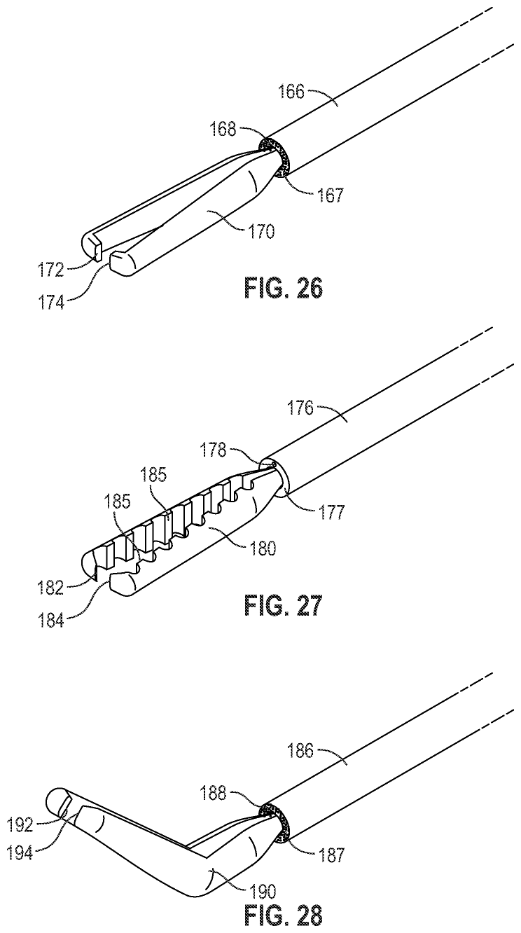

FIG. 26 shows intraocular portion 166 having vitreoretinal cutter or forceps 170, in accordance with yet another embodiment of the present invention. Here, intraocular portion 166 presents distal end 167. At distal end 167, intraocular portion 166 encompasses light ring 168 having a plurality of lights. Intraocular portion 166 includes vitreoretinal cutter 170. Vitreoretinal cutter 170 encompasses L-shaped forceps-like mechanism for holding and/or cutting of membrane in eye 14. Vitreoretinal cutter 170 includes first blade 172 and second blade 174. Here, first blade 172 indicates a L-shaped blade having positive pole for cauterization and second blade 174 indicates a L-shaped blade having negative pole for cauterization. Here, vitreoretinal cutter 170 draws power from power source and a light source 131 and helps to hold and/or cut membrane in eye 14 during the vitreoretinal and ocular surgery.

FIG. 27 shows intraocular portion 176 having vitreoretinal cutter or forceps 180 used for holding/grabbing tissue/membrane in eye 14, in accordance with yet another embodiment of the present invention. Here, intraocular portion 176 presents distal end 177. At distal end 177, intraocular portion 176 encompasses light or laser 178. Intraocular portion 176 includes vitreoretinal cutter 180. Vitreoretinal cutter 180 encompasses forceps-like mechanism i.e., serrated forceps for holding and/or cutting of membrane in eye 14. Vitreoretinal cutter 180 includes first blade 182 and second blade 184. Here, first blade 182 has a positive pole for cauterization and second blade 184 has a negative pole for cauterization. Each of first blade 182 and second blade 184 have teeth 185 (serrated forceps) to firmly grip the membrane during vitreoretinal and ocular surgery.

FIG. 28 shows intraocular portion 186 having vitreoretinal cutter 190, in accordance with yet another embodiment of the present invention. Here, intraocular portion 186 presents distal end 187. At distal end 187, intraocular portion 186 encompasses light ring 188. Intraocular portion 186 includes vitreoretinal cutter 190. Vitreoretinal cutter 190 encompasses angled forceps for holding and/or cutting of membrane in eye 14. Vitreoretinal cutter 190 includes first blade 192 and second blade 194. Here, first blade 192 indicates an angled blade having positive pole for cauterization and second blade 194 indicates an angled blade having a negative pole for cauterization. Here, vitreoretinal cutter 190 draws power from power source and a light source 131 and helps to hold and/or cut membrane in eye 14 during the vitreoretinal and ocular surgery.

The presently disclosed vitreoretinal cutter provides for cutting or peeling of membranes and cauterization at the same time. This reduces the time of surgery and likelihood of complications as this limits the number of entries each instrument enters and exits the eye during the vitreoretinal and ocular surgery.

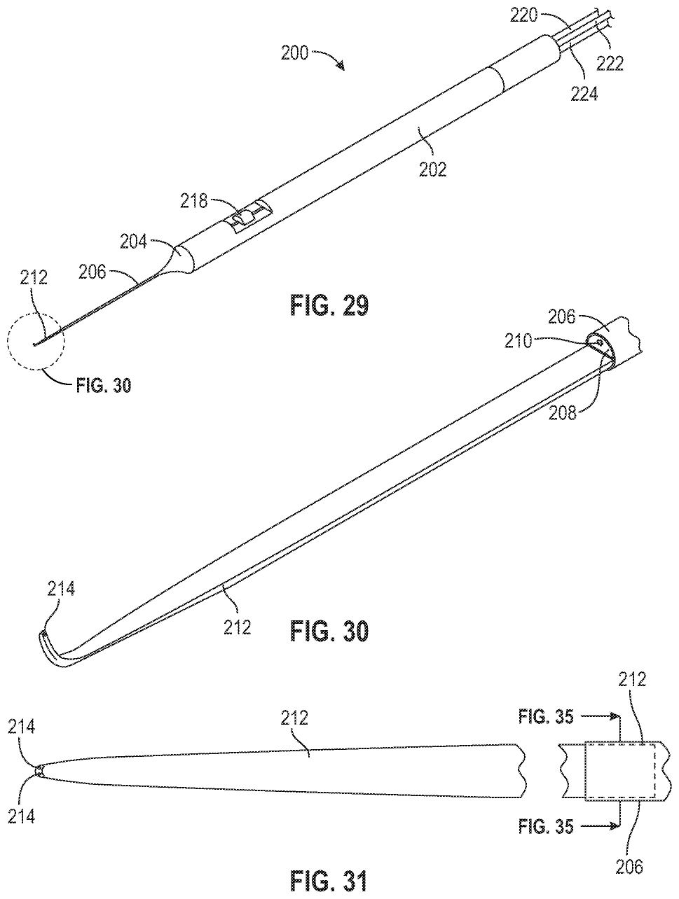

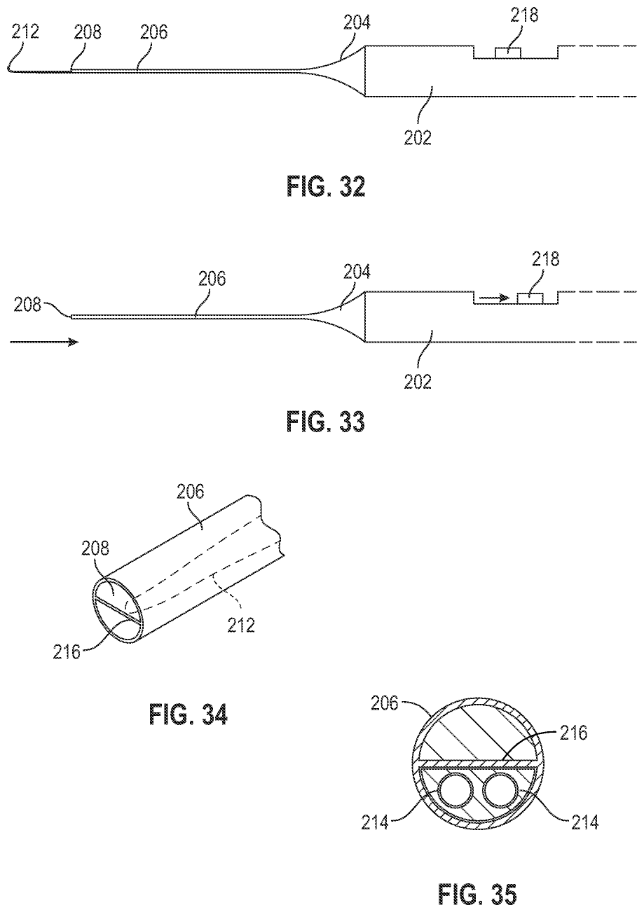

Surgical apparatus 12 further includes an intraocular pick and dissector. FIG. 29 shows a perspective view of intraocular pick and dissector 200, in accordance with one embodiment of the present invention. Intraocular pick and dissector 200 includes elongated tube or handle 202. Elongated tube 202 encompasses tapered section 204 at one end. Intraocular pick and dissector 200 includes shaft 206 extending from tapered section 204 of elongated tube 202. Shaft 206 presents distal end 208. In one example, shaft 206 includes light 210 such as Light Emitting Diode (LED) at distal end 208. Alternatively, shaft 206 includes a camera (not shown) for capturing and providing a live feed to aid the ophthalmologist during the surgery. In one implementation, intraocular pick and dissector 200 encompasses pick 212 that extends and retracts into shaft 206. FIGS. 30 and 31 show a perspective and a top view of pick 212 extending from shaft 206. Pick 212 encompasses injector ports 214 at the end as shown in at least FIGS. 30 and 31. In the present embodiment, shaft 206 encompasses pick receiving area 216 (FIG. 35) configured for receiving pick 212. Here, pick 212 extends from shaft 206 (FIG. 32) and retracts into pick receiving area 216 (FIG. 33). In order to extend or retract pick 212, elongated tube 202 presents button 218 that slides and operates pick 212. Upon engaging (FIG. 33), pick 212 retracts into pick receiving area 216 as shown in FIGS. 34 and 35. Further, elongated tube 202 includes fluid connecting port 220, light port 222 and power receiving port 224 at the other end (i.e., opposite end of tapered section 204).

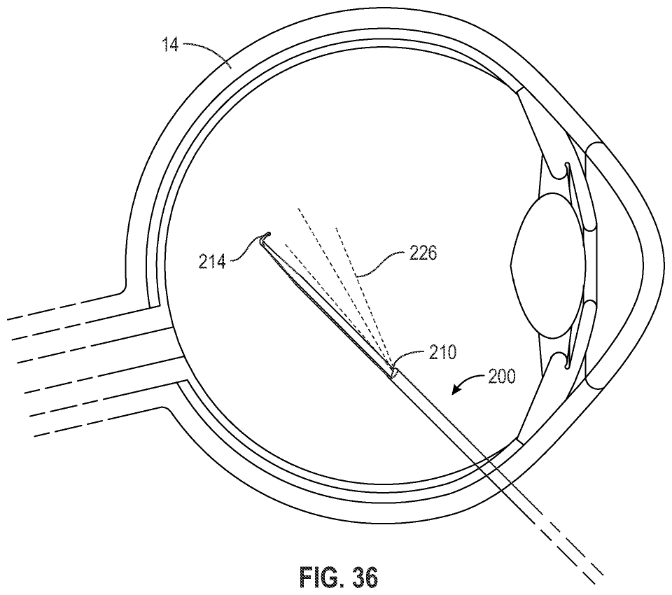

FIG. 36 shows a feature of intraocular pick and dissector 200 placed in eye 14. Here, intraocular pick and dissector 200 inserts in eye 14. Upon placing intraocular pick and dissector 200, light 210 emits light 226 for aiding the ophthalmologist during the vitreoretinal and ocular surgery. The ophthalmologist engages elongated tube 202 to extend or retract pick 212 into shaft 206 during vitreoretinal and ocular surgery.

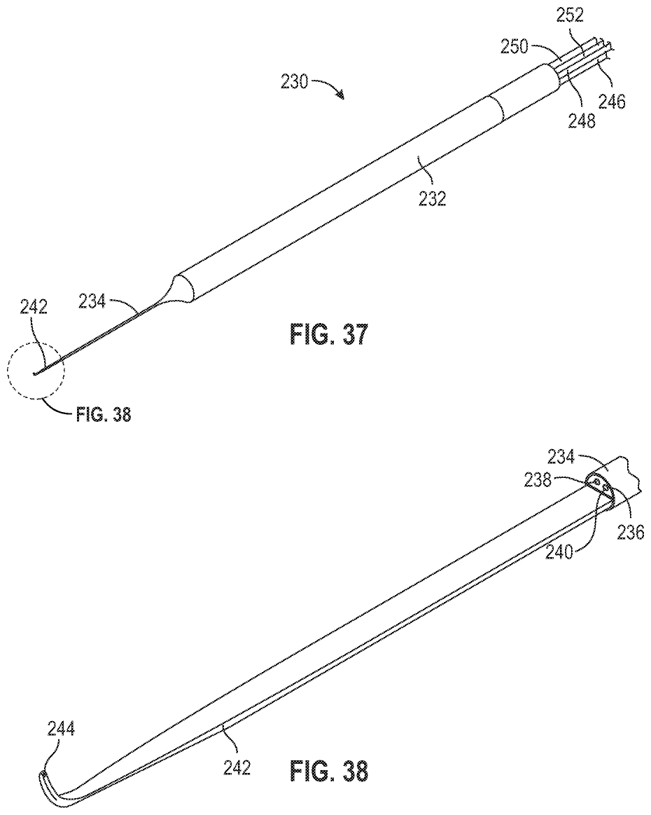

FIG. 37 shows a perspective view of intraocular pick and dissector 230, in accordance with another embodiment of the present invention. Intraocular pick and dissector 230 includes elongated tube or handle 232. Elongated tube 232 encompasses shaft 234 extending from elongated tube 232. Shaft 234 presents distal end 236. In one example, shaft 234 includes light 238 and laser 240 at distal end 236. FIG. 38 shows the feature of shaft 234 having light 238 and laser 240 at distal end 236. As explained above, intraocular pick and dissector 230 encompasses pick 242 that extends and retracts into shaft 234. Pick 242 encompasses injector ports 244 at the end, as shown in FIG. 38. Further, elongated tube 232 includes fluid connecting port 246, light port 248, laser port 250 and power receiving port 252 at the other end (i.e., opposite end of tapered section 234), as shown in FIG. 37.

The presently disclosed intraocular pick and dissector helps to pick and dissect highly vascularized tissue, membranes or scar tissue at the same time during the vitreoretinal and ocular surgery. This limits the number of entries of instruments used during the surgery. Limiting the number of entries of instruments helps to reduce potentially irreversible damage to the eye and increase the speed of the surgery. This greatly reduces the inconvenience to the patient undergoing the surgery.

Based on the above, it is evident that the presently disclosed surgical apparatus provides instruments that are multifunctional and reduces the number of times instruments enter in and out of the eye during the vitreoretinal and ocular surgery. This ensures faster and safer surgery.

In the above description, numerous specific details are set forth such as examples of some embodiments, specific components, devices, methods, in order to provide a thorough understanding of embodiments of the present disclosure. It will be apparent to a person of ordinary skill in the art that these specific details need not be employed, and should not be construed to limit the scope of the disclosure.

In the development of any actual implementation, numerous implementation-specific decisions must be made to achieve the developer's specific goals, such as compliance with system-related and business-related constraints. Such a development effort might be complex and time consuming, but is nevertheless a routine undertaking of design, fabrication, and manufacture for those of ordinary skill. Hence, as various changes could be made in the above constructions without departing from the scope of the invention, it is intended that all matter contained in the above description or shown in the accompanying drawings shall be interpreted as illustrative and not in a limiting sense.

The foregoing description of embodiments is provided to enable any person skilled in the art to make and use the invention. Various modifications to these embodiments will be readily apparent to those skilled in the art, and the novel principles and invention disclosed herein may be applied to other embodiments without the use of the innovative faculty. The invention set forth in the description is not intended to be limited to the embodiments shown herein, but is to be accorded the widest scope consistent with the principles and novel features disclosed herein. It is contemplated that additional embodiments are within the spirit and true scope of the disclosed invention.

What is claimed is:

1. A surgical apparatus for performing a microsurgery, said surgical apparatus comprising:
a vitreous cutter comprising a handle, wherein said handle comprises a suction tube at one end and a shaft at another end, wherein said shaft comprises a cutting port, and wherein said shaft comprises a light and/or a laser at its distal end;
a vitreoretinal surgical tool comprising a vitreoretinal cutter; and
a cannula comprising fenestrations at its distal end, wherein said cannula intraocular portion receives fluid and dispenses the fluid through said fenestrations lessening the flow at an infusion site in an eye,
wherein said cutting port cuts vitreous into smaller pieces or said laser liquefies the vitreous, wherein said shaft receives the cut vitreous pieces and said suction tube draws out the cut vitreous pieces from the eye, and wherein said light aids in viewing the vitreous during cutting of the vitreous, and
wherein said vitreoretinal cutter holds and/or cuts a membrane in the eye during the microsurgery.

2. The surgical apparatus of claim 1, wherein said cannula comprises an intraocular portion, wherein said intraocular portion connects to an infusion tube, wherein said intraocular portion receives fluid through said infusion tube and dispenses the fluid through said fenestrations.

3. The surgical apparatus of claim 1, wherein the microsurgery is selected from the group consisting of Rhegmatogenous Retinal Detachment, Macular Holes, Epiretinal Membranes, Retinal Transplantation, Dislocated intraocular lens (IOL), Non-Clearing Vitreous Hemorrhage, Proliferative Diabetic Retinopathy, Traction Retinal Detachment, Retinopathy of Prematurity, Pediatric Rhegmatogenous Retinal Detachment, Uveitis induced Retinal Detachment, Choroidal and Retinal Biopsy, Giant Retinal Tears, Choroidal Hemorrhage, Submacular Hemorrhage, Age-Related Macular Degeneration, Uveal Effusion Syndrome, Endophthalmitis, Intraocular Foreign Body, Open Globe rupture, Retinoschisis Retinal Detachment, Optic Pit Maculopathy, Retinal Detachment, and Proliferative Vitreoretinopathy.

4. The surgical apparatus of claim 2, wherein said intraocular portion of said cannula comprises a tapered tip or curved tip.

5. The surgical apparatus of claim 1, wherein each of said fenestrations comprises an angled opening to distribute the flow of said fluid.

6. The surgical apparatus of claim 1, wherein said vitreous cutter operates using spring-driven mechanisms or dual pneumatic pumps that independently control the opening and closing of said cutter port.

7. The surgical apparatus of claim 1, wherein said shaft is provided in one of straight configuration, bent configuration and curved configuration.

8. The surgical apparatus of claim 1, wherein said cutting port has a size of 19-gauge or smaller.

9. The surgical apparatus of claim 1, wherein said vitreoretinal cutter has a scissor-like mechanism for holding and/or cutting the membrane in the eye.

10. The surgical apparatus of claim 1, wherein said vitreoretinal cutter has a forceps-like mechanism for holding and/or cutting the membrane in the eye.

11. The surgical apparatus of claim 1, further comprises an intraocular pick and dissector for picking up the membrane or scar tissue in the eye.

12. The surgical apparatus of claim 11, wherein said intraocular pick and dissector comprises a shaft, wherein said shaft comprises a pick at its distal end.

13. The surgical apparatus of claim 12, wherein said pick extends and retracts into said shaft.

14. The surgical apparatus of claim 13, wherein said intraocular pick and dissector comprises a button configured to slide for extending and retracting said pick into said shaft.

15. A method of providing a surgical apparatus for performing a microsurgery, the method comprising steps of:
providing a vitreous cutter comprising a handle, said handle comprising a suction tube at one end and a shaft at another end, said shaft comprising a cutting port, said shaft comprising a light and a laser at its distal end;
cutting a vitreous into smaller pieces using said cutting port or liquefying the vitreous using said laser, said shaft receiving the cut vitreous pieces and said suction tube drawing out the cut vitreous pieces from the eye;
viewing cutting of the vitreous with the help of said light;
providing a vitreoretinal surgical tool comprising a vitreoretinal cutter; and
holding and/or cutting a membrane in the eye during the microsurgery using said vitreoretinal cutter.

16. The method of claim 15, further comprising:
providing a cannula comprising an intraocular portion, said intraocular portion connecting to an infusion tube, and said intraocular portion comprising fenestrations at its distal end; and
receiving fluid at said intraocular portion through said infusion tube and dispensing the fluid through said fenestrations for lessening the flow at an infusion site in an eye.

17. The method of claim 15, wherein the microsurgery is selected from the group consisting of Rhegmatogenous Retinal Detachment, Macular Holes, Epiretinal Membranes, Retinal Transplantation, Dislocated intraocular lens (IOL), Non-Clearing Vitreous Hemorrhage, Proliferative Diabetic Retinopathy, Traction Retinal Detachment, Retinopathy of Prematurity, Pediatric Rhegmatogenous Retinal Detachment, Uveitis induced Retinal Detachment, Choroidal and Retinal Biopsy, Giant Retinal Tears, Choroidal Hemorrhage, Submacular Hemorrhage, Age-Related Macular Degeneration, Uveal Effusion Syndrome, Endophthalmitis, Intraocular Foreign Body, Open Globe rupture, Retinoschisis Retinal Detachment, Optic Pit Maculopathy, Retinal Detachment, and Proliferative Vitreoretinopathy.

18. The method of claim 15, further comprising providing a tapered tip or curved tip at the distal end of said intraocular portion of said cannula.

19. The method of claim 15, further comprising operating said vitreous cutter using spring-driven mechanisms or dual pneumatic pumps that independently control the opening and closing of said cutter port.

20. The method of claim 15, further comprising providing an intraocular pick and dissector for picking up the membrane or scar tissue in the eye, said intraocular pick and dissector comprising a shaft having a pick at its distal end, said pick extending and retracting into said shaft.
